(19) United States
(12) Patent Application Publication
(10) Pub. No.: US 2025/0332362 A1
(43) Pub. Date: Oct. 30, 2025

(54) MATERIALS WITH SURFACE MICROSTRUCTURES, METHODS OF MAKING AND USING THE SAME

(71) Applicant: University of Iowa Research Foundation, Iowa City, IA (US)

(72) Inventors: Yuliang Xie, Coralville, IA (US); Connor Grierson, Iowa City, IA (US); Garret W. Caltrider, Iowa City, IA (US)

(21) Appl. No.: 19/170,662

(22) Filed: Apr. 4, 2025

Related U.S. Application Data
(60) Provisional application No. 63/575,468, filed on Apr. 5, 2024.

(51) Int. Cl. A61M 16/00; A61M 16/04

(52) U.S. Cl. CPC A61M 16/0006; A61M 16/0402

(57) ABSTRACT

Described herein are medical devices and methods of using and making the same. The medical devices include a substrate, and a plurality of microstructures provided on a surface of the substrate. When actuated by vibrational or acoustic energy, they facilitate movement of fluids along a surface of the medical device.

### CROSS REFERENCE TO RELATED APPLICATION

This application claims the benefit of U.S. Provisional Application No. 63/575,468, filed Apr. 5, 2024, the entire contents of which are incorporated by reference herein.

### FEDERALLY SPONSORED RESEARCH AND DEVELOPMENT

This invention was made with government support under Grant EB033395 awarded by the National Institutes of Health. The government has certain rights in the invention.

### BACKGROUND OF THE INVENTION

Mucus contacted medical devices, such as airway devices and eye prostheses, suffer from mucus accumulation. Plugged mucus causes bacterial infections, airway blockages, and a requirement of frequent device cleanings and replacement, which adds significant care burdens for the patient and support community. Current approaches to mitigate mucus accumulation involve strong mechanical forces or medications, thus having intrinsic limitations and side effects.

There are numerous implanted/indwelling medical devices that directly contact mucus, including airway devices (e.g., tracheostomy tubes, endotracheal tubes, voice prostheses), and eye prostheses. In the United States, more than 100,000 tracheostomies are performed annually. In all of these types of implanted/indwelling medical devices, mucus accumulation can be a persistent and problematic issue.

Mucus persistently accumulates on device surfaces (FIGS. 1A and 1B), causing severe health consequences. First, accumulated mucus traps bacteria and causes infections. Infection is the leading safety concern when applying such devices. For example, although infections of ocular surfaces are rare, ocular prostheses bring patients a high and lifetime risk of infection. Such infections further stimulate inflammation and mucus discharge. These problems affect 93% of patients using prosthetic eye wear. In airways, the presence of a tracheostomy tube is a predisposing factor for bacterial tracheitis, where colonization of single and multi-bacterial species occurs on the tube in 95% and 83% of cases, respectively. Second, in airway devices, accumulated mucus blocks the respiratory tract, causing difficulty breathing. Third, a requirement for frequent mucus cleaning adds significant care burdens for the patient and support community.

To date, various approaches have been proposed to clear mucus from medical devices. Suctioning is by far the most common way that patients clean airway devices. In that procedure, a catheter with negative air pressure is introduced into the device tube to remove mucus. Although suctioning has been used for decades, strong suction pressure causes side effects such as, mucosal trauma, hypoxemia, bronchospasm, infection, and in some instances even lung collapse. In the 2000s, catheter balloons, mucus shavers, and mucus slurpers were developed. The devices use a catheter-guided object sweeping across the luminal surface of airway devices to replace negative pressure in suction. However, repeated use of these mechanical devices and the procedure for using the same can introduce pain, risk of infection, and add burden for the patient due to complexity of equipment operation and maintenance.

Mucolytics are common medications that are used, through oral or topical application, to thin mucus that has accumulated on the device. However, repeated use of mucolytics stimulate mucosal surfaces, causing nausea, runny nose, sore throat, and drowsiness. Antibiotics, antibacterials and antimicrobials are also commonly used on patients with airway devices or eye protheses. Although effective in bacterial elimination, the increased use of broad-spectrum antimicrobials has resulted in the increased bacteria resistance and recovery of mycobacteria, yeast, and fungi, which are more difficult to treat.

Methods of developing ways to reduce mucus adhesion on surfaces have also been pursued. For example, hydrophilic polymer coated surfaces (e.g., polyethylene glycol, basic and zwitterionic, hydroxyls, acids, and amines) exhibit minimum adhesion to porcine mucus. The low mucus adhesion derives mainly from surface-water interactions (polar and hydrogen-bonding) and possibly the flexibility of graft polymer chains. Low mucus attaching surfaces have also been studied on mucus penetrating nanoparticles, and mucus-bacteria surface interactions. Although holding promise, these surfaces lack a mechanism to actively remove mucus. Without mucus clearance, mucus soon accumulates, making a cleaning procedure inevitable. Considering the vast number of mucus contacted medical devices, high health risk of mucus accumulation, and limitations of current mucus cleaning methods, there is an urgent un-met need to develop new solutions for effectively clearing mucus from medical devices.

### SUMMARY OF THE INVENTION

Various aspects of the disclosure are directed towards medical devices comprising a substrate, and a plurality of microstructures provided on a surface of the substrate. Various non-limiting aspects of the disclosure can be described as follows.

In some instances, a first aspect of the disclosure can be described as a medical device comprising a substrate, and a plurality of microstructures provided on a surface of the substrate, where the plurality of microstructures provide the medical device with a microstructured surface.

In some instances, a second aspect of the disclosure can be described as a medical device according to the first aspect, wherein the substrate is made of a first polymeric material.

In some instances, a third aspect of the disclosure can be described as a medical device according to the first or second aspect, wherein the plurality of microstructures is made of a second polymeric material.

In some instances, a fourth aspect of the disclosure can be described as a medical device according to any one of the first through third aspects, wherein the substrate is made of a polymeric sheet.

In some instances, a fifth aspect of the disclosure can be described as a medical device according to any one of the first through fourth aspects, wherein the substrate is in the shape of a tube, a flat surface, a curved surface, or a sphere.

In some instances, a sixth aspect of the disclosure can be described as a medical device according to any one of the first through fifth aspects, wherein some or all of the plurality of microstructures are asymmetrical, as indicated by dissecting one of the microstructures with a vertical plane extending through a center point of the microstructure.

In some instances, a seventh aspect of the disclosure can be described as a medical device according to any one of the first through sixth aspects, wherein a width or diameter of some or all of the plurality of microstructures ranges from about 25 nm to about 100 μm.

In some instances, an eighth aspect of the disclosure can be described as a medical device according to any one of the first through sixth aspects, wherein a width or diameter of some or all of the plurality of microstructures ranges from about 100 μm to about 500 μm.

In some instances, a ninth aspect of the disclosure can be described as a medical device according to any one of the first through eighth aspects, wherein the length of some or all of the plurality of microstructures ranges from about 200 μm to about 2000 μm.

In some instances, a tenth aspect of the disclosure can be described as a medical device according to any one of the first through ninth aspects, wherein the length of some or all of the plurality of microstructures ranges from about 40 μm to about 60 μm.

In some instances, an eleventh aspect of the disclosure can be described as a medical device according to any one of the first through tenth aspects, wherein the plurality of microstructures further comprises a hydrophilic coating thereon.

In some instances, a twelfth aspect of the disclosure can be described as a medical device according to the eleventh aspect, wherein the coating is made of a polyethyleneimine, a polyvinyl alcohol, or a polyethylene glycol.

In some instances, a thirteenth aspect of the disclosure can be described as a medical device according to any one of the of the first through twelfth aspects, wherein the plurality of microstructured pillars are polarized.

In some instances, a fourteenth aspect of the disclosure can be described as a medical device according to the fourteenth aspect, wherein the plurality of microstructured pillars are asymmetric.

In some instances, a fifteenth aspect of the disclosure can be described as a medical device according to any one of the first through the fourteenth aspects, wherein the medical device is associated with a surface of a second medical device.

In some instances, a sixteenth aspect of the disclosure can be described as a medical device according to the fifteenth aspect, wherein the second medical device is a tube.

In some instances, a seventeenth aspect of the disclosure can be described as a method of removing a fluid from a surface of a medical device according to any one of the first through sixteenth aspects, wherein the method comprises subjecting the medical device to an energy input to vibrate the plurality of microstructures, wherein vibration of the microstructures induces a flow of a fluid located on the microstructured surface.

In some instances, an eighteenth aspect of the disclosure can be described as a method according to the seventeenth aspect, wherein the fluid is mucus.

In some instances, a nineteenth aspect of the disclosure can be described as a method according to the seventeenth or eighteenth aspect, wherein the energy input is acoustic.

In some instances, a twentieth aspect of the disclosure can be described as a method according to any one of the seventeenth through the nineteenth aspects, wherein the medical device is configured for insertion in a body part of a mammal.

In some instances, a twenty-first aspect of the disclosure can be described as a method according to the twentieth aspect, wherein the body part is a trachea.

### BRIEF DESCRIPTION OF DRAWINGS

FIG. 1A and FIG. 1B are prior art images of mucus build up and plugging in an endotracheal tube (top) and a tracheostomy tube (bottom).

FIG. 2A and FIG. 2B are prior art fluorescence microscopy images illustrating clearance of mucus strands on non-cystic fibrosis (non-CF) (top) and cystic fibrosis (CF) (bottom) pig airways.

FIG. 3A and FIG. 3B are prior art images showing ciliary microstructures fabricated with anode aluminum oxide (AAO) templates (scale bar=1 μm).

FIG. 4A and FIG. 4B provide prior art images showing ciliary microstructures that can be polarized by Janus gold deposition. FIG. 4A is a prior art image of microstructures undergoing a conversion from straight nanopillars to uniformly bent Janus nanopillar. FIG. 4B is a prior art SEM image displaying the bent nanopillar (scale bar=500 nm).

FIGS. 5A through 5E provide a schematic illustration showing an exemplary template-based method to fabricate polarized ciliary microstructures. The polarized ciliary microstructures can be made using the following steps: template provision (FIG. 5A), spincoat prepolymer (FIG. 5B), polymerization (FIG. 5C), template removal (FIG. 5D), and Janus gold deposit (FIG. 5E).

FIG. 6 provides prior art images (FIGS. 6A, 6B and 6C) of 3D printed products with a multi-scale projection stereolithography (MPS) system (FIGS. 6A and 6C scale bars=500 μm).

FIG. 7 is a prior art schematic illustration of an MPS system for high-resolution, scalable 3D printing.

FIGS. 8A through 8D provide a schematic illustration showing exemplary methods to polarize microstructures with 3D printing.

FIG. 9 is a measurement of (FIG. 9A) friction and (FIG. 9B) detachment between mucus and surface.

FIGS. 10A and 10B are prior art images showing acoustic waves vibrating microstructures and perturbing surrounding fluids for mixing in channels (scale bar=200 μm).

FIG. 11 is a prior art image showing acoustic waves vibrating polarized microstructures to pump fluids (scale bar=250 μm).

FIG. 12 is a graphical display illustrating attenuation coefficient in tissues.

FIG. 13 is a schematic illustration showing exemplary in vivo manipulation of particles.

FIG. 14 is a schematic illustration showing an exemplary platform to test acoustic actuated mucociliary transport (MCT) in vitro.

FIGS. 15A-15C provide a schematic illustration showing a test of a tracheal tube with a pig exhibiting CF in vivo.

FIGS. 16A-16D show SEM images of exemplary engineered polydimethylsiloxane (PDMS) microstructures.

FIG. 17 provides schematic illustrations showing development (FIG. 17A) and acoustic actuation (FIG. 17B) of PDMS microstructures to direct flow, and time-elapsed fluorescence images showing net flow around acoustically actuated round-shaped pillars (FIG. 17C) and net flow around teardrop structures (FIG. 17D) (FIGS. 17C and 17D scale bars=100 μm).

FIG. 18 is a time elapsed fluorescence imaging showing net flow around teardrop shaped pillar structures.

FIG. 19 is a time elapsed fluorescence imaging showing net flow around semicircular or “letter D” shaped pillar structures.

FIG. 20 provides schematic illustrations of additional exemplary asymmetric pillar microstructures.

FIG. 21 provides images of 3D-printed tilted, cilia like structures(scale bar is scale=1 mm.

FIGS. 22A and 22B provides images of 3d-printed tilted, cilia-like structures that drive directional motion on a glass slide.

FIGS. 23A-23E provides schematic illustrations of additional exemplary microstructures structures (scale bar=500 μm.)

FIGS. 24A-24C is a collection of SEM images of a microfabricated PDMS structure, FIG. 24A has a 1.00 mm scale, FIG. 24B has a 300 μm scale, and FIG. 24C has a 50.0 μm scale.

FIG. 25A is a schematic of fluid being driven by asymmetric, microfabricated PDMS structures.

FIG. 25B and FIG. 25C are fluorescent images of fluid driven by asymmetric, microfabricated PDMS structures.

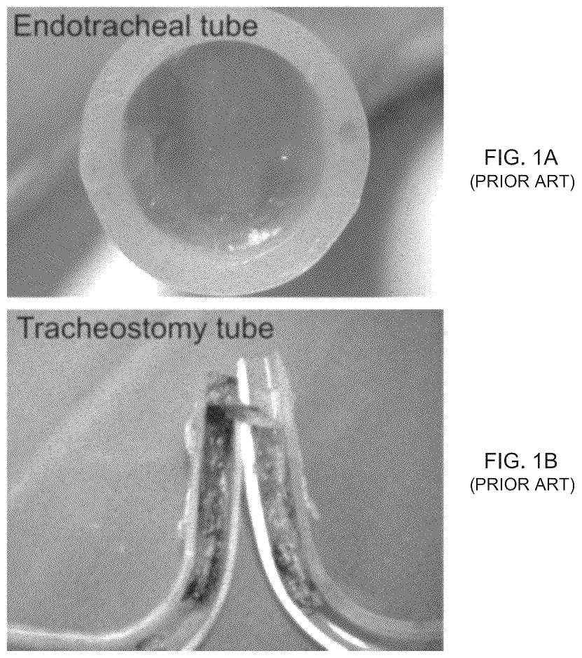

### DETAILED DESCRIPTION

There are an enormous number of indwelling medical devices that directly contact mucus, including tracheostomy tubes, endotracheal tubes, voice prostheses, and eye prostheses. A leading health risk with such devices stems from mucus accumulation. Mucus plugging traps bacteria, leading to chronic infections. Mucus accumulating devices require frequent cleaning or replacement, adding burdens to the patient and the support community. In airway devices, mucus occlusion blocks the respiratory tract, causing difficult breathing. Despite the severe health risks associated with mucus accumulation, current methods to mitigate mucus accumulation (e.g., suction, shaver, slurper, balloon, mucolytics, antibiotics, and low mucus adhesion surface) involve strong force or medication, thus have intrinsic limitations and side effects. Therefore, there is an urgent un-met need for development of mechanisms to clean mucus from medical devices.

Mucociliary transport (MCT), a process by which waves of beating cilia move a blanket of mucus, forms the first-line barrier against infection in respiratory and genital tracts. In the respiratory tract, MCT clears mucus which traps inhaled pathogens to keep the lung sterile; in the female genital tract, MCT not only protects against infection, but also regulates sperm transportation and fertilization. Inspired by the effectiveness of MCT in clearing mucus, various aspects of this disclosure are directed to the fabrication of engineered surfaces that enable MCT function. Engineered surfaces that enable MCT function are achieved through a combination of cilia fabrication, surface modification, and acoustic actuation with the following aims. Some aspects of the present disclosure are directed to fabricating engineered surfaces with polarized ciliary structures. Some aspects of the disclosure are directed to tailoring engineered surfaces for MCT based upon the viscosity and/or tackiness of different types of mucus. Some aspects of the disclosure are directed to the development of platforms, such as banana slug and pig models, to test acoustically actuated MCT on engineered surfaces in vitro and in vivo. Recapitulating ciliary microstructure, surface chemistry, polarization, and beating will allow mucus movement across engineered MCT surfaces according to various aspects of the disclosure. Acoustic waves provide driving forces for mucus movement over said engineered MCT surfaces.

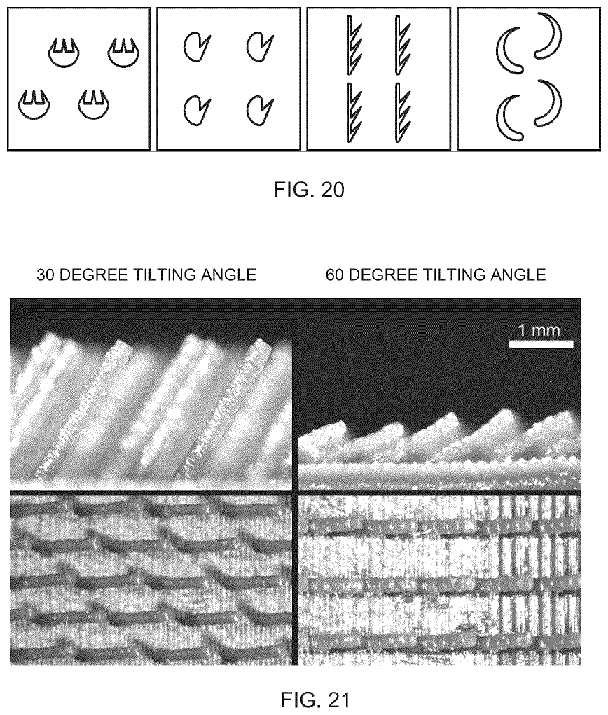

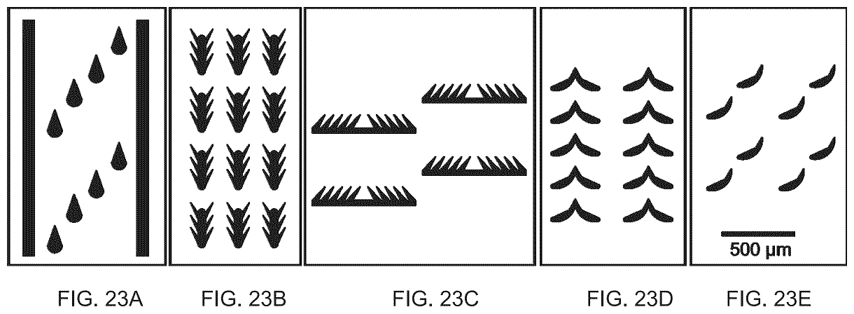

Various aspects of the disclosures pertain to polymers comprising microstructures (also referred to herein as a microstructured polymer). In some instances, the microstructures are integrally formed with a surface of a polymeric substrate such as a polymer sheet, bead, sphere, granule, or other shaped polymer. In some instances, the microstructures are applied onto a surface of a polymeric substrate such as a polymer sheet, bead, sphere, granule, or other shaped polymer. In some instances, the microstructures are nanopillars. In some instances, the microstructures are asymmetrical, as shown by dissecting the microstructure with a vertical plane extending through a center point of the microstructure. In some instances, the microstructures are ciliary structures. In some instances, the microstructures are complex geometries. In some instances, the microstructures are shaped liked stars. In some instances, the microstructures are shaped like tear drops. In some instances, the microstructures are shaped like circles. In some instances, the microstructures are shaped like squares. In some instances, the microstructures are shaped like rectangles. In some instances, the microstructures are shaped like letters, such as, for example, the letter “D.” In some instances, the microstructures are shaped like those in FIG. 20. In some instances, the microstructures are shaped like those in FIG. 23A-23E. In some instances, the microstructures are Janus nanopillars. In some instances, a diameter of the microstructure is about 50 to about 500 nm. In some instances, a diameter microstructure is about 25 to about 100 nm. In some instances, a diameter of the microstructure is about 50 to about 150 nm. In some instances, a diameter of the microstructure is about 200 to about 300 nm. In some instances, a diameter of the microstructures is about 1 μm to about 500 μm. In some instances, a diameter of the microstructures is about 100 μm to about 300 μm. In some instances, a diameter of microstructures is about 1 μm to about 100 μm. In some instances, a diameter of the microstructure is about 1 um to about 50 μm. In some instances, a diameter of the microstructure is about 5 to about 100 μm. In some instances, a diameter of the microstructure is about 5 to about 30 μm. In some instances, a width of the microstructures is about 30 to about 100 μm. In some instances, a width of the microstructure is about 1 to about 200 μm. In some instances, the a of the microstructures is about 5 to 100 μm. In some instances, the width of the microstructures is about 5 μm to about 500 μm. In some instances, the width of the microstructures is about 100 μm to about 500 μm. In some instances, the width of the microstructures is about 50 μm to about 150 μm. In some instances, a length of the microstructure can be about 25 μm to about 75 μm. In some instances, a length of the microstructure is about 40 μm to about 60 μm. In some instances, a length of the microstructure is about 50 μm. In some instances, a length of the microstructure is about 100 μm to about 300 μm. In some instances, a length of the microstructure is about 200 μm. In some instances, a length of the microstructure is about 200 to 2000 μm. In some instances, a length of the microstructure is about 200 to 1000 μm. In some instances, a length of the microstructure is about 1000 to about 2000 μm. In some instances, a length of the microstructure is about 100 μm to about 3000 μm. In some instances, a length of the microstructure is about 1000 μm to about 3000 μm. In some instances, the microstructures or microstructured polymer can be polarized. In some instances, the microstructure or microstructured polymer is polarized via Janus gold deposition. In some instances, the microstructures or microstructured polymer are coated. In some instances, the microstructures or microstructured polymer are coated with gold. In some instances, the microstructures are coated with silver.

Various aspects of the disclosure pertain to microstructured polymers for medical applications. In some instances, microstructured polymers according to the disclosure can have a configuration of a medical device. In some instances, microstructured polymers according to the disclosure can be in the form of a flat or curved sheet, or in the form of spheres or beads, that can be used alone or applied to a surface of a medical device. In some instances, microstructured polymers according to the disclosure are integrated into the medical device. In some instances, a sheet of a microstructured polymer is applied to the medical device. In some instances, the medical device is a tube. In some instances, the medical device is a tracheal tube. In some instances, the microstructured polymer is in the form of a cylinder. In some instances, the microstructured polymer is in the form of a hollow entity. In some instances, the microstructured polymer is in the form of a hollow cylinder. In some instances, the microstructured polymer is in the form of a hollow entity that comprises the microstructures on an inner surface of the hollow entity.

Various polymers may be used in the fabrication of polymers comprising microstructures according to the disclosure. In some instances, the polymer comprises, consists essentially of, or consists of a natural polymer. In some instances, the polymer comprises, consists essentially of, or consists of a synthetic polymer. In some instances, the polymer comprises, consists essentially of, or consists of a combination of natural polymers and synthetic polymers. In some instances, the polymer comprises, consists essentially of, or consists of one or more of polyethylene, polypropylene, polystyrene, polyvinyl chloride, synthetic rubber, phenol formaldehyde resin, neoprene, nylon, polyacrylonitrile, PVB, and silicones. In some instances, the polymer comprises, consists essentially of, or consists of poly(ethylene glycol) diacrylate (PEGDA). In some instances, the polymer comprises, consists essentially of, or consists of polydimethylsiloxane (PDMS).

In accordance with various aspects of the disclosure, microstructures are provided on a surface of a polymeric substrate having a structure. Exemplary polymeric substrate structures include, but are not limited to shaped sheets, strips, beads, spheres, and granules. The microstructures are designed to facilitate the movement, or clearance, of viscous fluids such as mucus from the surface of the polymeric substrate. In some instances, the microstructures are casted onto the polymeric substrate. In some instances, the microstructures are casted from a template-based method. In some instances, the template is self-ordered porous anodized aluminum oxide (AGO). In some instances, the template determines the diameter or width of the microstructures. In some instances, the template determines the length of the microstructures. In some instances, the microstructures are 3D printed onto the polymeric substrate. In some instances, the polymeric substrate is 3D printed. In some instances, the polymeric substrate and microstructures are integrally 3D printed. In some instances, the 3D printing can use a multi-scale projection stereolithography (MPS). In some instances, an MPS system can be used to 3D print medical devices with microstructures. In some instances, an MPS system can be used to fabricate microstructures with different stiffnesses and surface properties. In some instances, the microstructures are microfabricated.

In some instances, the surfaces of the microstructures are modified to more closely relate to MCT. In some instances, the surfaces of the microstructures are coated with a hydrophilic material. Suitable hydrophilic materials include, but are not limited to, polyethyleneimines, polyvinyl alcohols, polyethylene glycols and glycans. In some instances, the surfaces of the microstructures are coated with a polyethylene glycol (PEG). In some instances, the surfaces of the microstructures are coated with a glycan. In some instances, the surfaces of the microstructures are coated to reduce the tendency of mucus to stick or adhere to the surfaces of the microstructures.

In instances, vibrations are applied to microstructured polymers according to the disclosure to aid in the movement of mucus through the microstructured polymers. In some instances, the applied vibrations are free vibrations or naturally-occurring vibrations. In some instances, the applied vibrations are forced vibrations. In some instances, the applied vibrations are damped vibrations. In some instances, the applied vibrations are acoustic vibrations. In some instances, the acoustic vibrations are generated via acoustic energy (waves). In some instances, the applied vibrations are a combination of one or more of naturally-occurring vibrations, forced vibrations, damped vibrations, and acoustic vibrations. In some instances, the acoustic energy can penetrate hydrogel, human tissue, or tissue-device interfaces.

In instances, microstructured polymers according to the disclosure are used to treat mucus buildup in patients. In some instances, the microstructured polymers are inserted into a patient that requires a tube inserted. In some instances, the microstructures of the microstructured polymers are used to break mucus build up within patients. In some instances, the mucus is broken up in response to the applied vibrations, facilitating movement of the mucus across the surface of the microstructured polymer.

### Examples

Fabricating Engineered Surfaces with Polarized Ciliary Structures.

According to various aspects of the disclosure, polymer (e.g., polydimethylsiloxane and poly(ethylene glycol) diacrylate) ciliary surfaces can be fabricated using anodized aluminum oxide as templates, and the ciliary structure can be polarized with a Janus gold coating. The polymer can be made in the form of, for example, a tracheal tube using standard 3D-printing technology, with a luminal side containing artificial “cilia” in the form of asymmetric structures. The template-based methods and 3D printing provide complementary capacities in the design of dimensions and materials for engineered MCT surfaces.

### Understanding Mucus Stickiness (Viscosity and Tackiness) on Engineered MCT Surfaces.

The surface chemistry of engineered surface ciliary structures can be modified and stickiness between mucus and the engineered surfaces can be evaluated with respect to friction (i.e., force against lateral motion) and detachment (i.e., force against vertical detachment) by tribo-rheometry. Polymer ciliary surfaces coated with hydrophilic molecules (e.g., poly(ethylene glycol) and glycan) will reduce mucus stickiness.

### Platforms to Test Acoustic Actuated MCT on Engineered Surfaces.

Mucus motion on an engineered surface of MCT can be assessed in vitro. Slug mucus and agar gel is used to mimic human mucus and tissue, respectively. Engineered surface ciliary structures are vibrated with acoustic waves, and mucus motion is directed by ciliary polarity. In one instance, an in vivo test for mucus clearance on a tracheal tube, with luminal surface of MCT, placed onto a pig trachea is conducted. A clinically applied ultrasound generator can be utilized to actuate MCT. The acoustic waves will penetrate hydrogels or animal tissues to move mucus on the engineered surface of MCT, therefore increasing mucus clearance. Such endeavors will provide for 1) revealing the mechanism of MCT on engineered surfaces and 2) delivering a prototype of tracheal tube with a luminal surface of MCT to reduce mucus accumulation, bacterial infection, and care burdens.

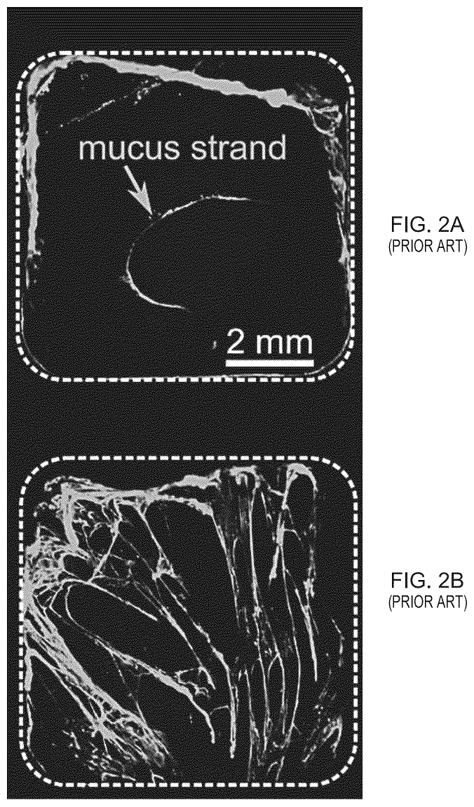

Mucociliary transport (MCT) is effective in clearing mucus. MCT is a process where waves of beating cilia move a blanket of mucus across a ciliary epithelium. MCT forms the first-line barrier against infections in many organs. In the respiratory tract, MCT clears mucus that traps inhaled pathogens to keep the lung sterile. Previous studies have investigated the mechanism of MCT on pig trachea, showing that after traveling up the submucosal gland duct, mucus emerges onto the airway surface in the form of strands. MCT is achieved by breaking strands and sweeping them across the surface to capture particulate material and pathogens (FIG. 2A). In cystic fibrosis (CF), mucus strands fail to break-free from submucosal glands, causing defective MCT and mucus accumulation (FIG. 2B). MCT also plays an important role in female genital tracts, where MCT not only protects against infection, but also regulates sperm transportation and fertilization. For example, MCT of cervical mucus cycles with menstruation, where effective transportation of sperm is only feasible over a few days; at other times of the cycle, mucus traps and clears sperm to prevent fertilization. The biological MCT inspires the conceptual basis for the presented engineered surface of MCT.

### Key Features of Biological MCT.

To perform MCT, the epithelium evolved a series of structural, biochemical, and biophysical features. First, MCT requires ciliary microstructures. Ciliated cells are the dominant epithelial cell type driving MCT. In airways, each ciliated cell contains ˜200 to 300 cilia on its apical surface; cilia are 0.2 to 0.3 μm in diameter and range in length from 6 to 7 μm in the upper airways to 4 μm in the smaller airways. These cilia provide the structural foundation for MCT. Second, MCT requires a ciliary surface that does not have a tendency to stick or adhere to mucus. In ciliated cells, each cilium is surrounded by a special cilia membrane, primarily composed of amphipathic lipids. In aqueous environments, the hydrophobic acyl groups associate with each other, and the polar heads associate with water to allow surface hydrophilicity. In addition, a large number of macromolecules (such as polysaccharides) are tethered on cilia surfaces, forming a brush-like structure. Thus, mucus cannot penetrate into the periciliary space, creating a distinct mucus-free layer (i.e., periciliary liquid, PCL). PCL prevents the epithelium from adhering to the overlying mucus and provides a low-viscosity environment for cilia stroke. Third, MCT also requires cilia beating to move mucus, and cilia polarization to direct mucus motion. For example, nasal cilia beating frequency values in 11-15 Hz and the beat pattern is asymmetric. If cilia actuation and direction is impaired in disease, MCT becomes dysfunctional. For example, patients with primary ciliary dyskinesia show dysfunctional cilia beat patterns (e.g., slow beating, low amplitude beating; and directionless motion). Table 1 below features biological and engineered surfaces of MCT.

TABLE 1

| | Biological surface | Engineered surface |
|---|---|---|
| Features | of MCT | of MCT |
| Ciliary structure | Cilia biogenesis | Microfabrication, |
| | | 3D printing |
| Low mucus | Tethered macromolecules | Surface chemical |
| adhesion | Periciliary liquid layer | modification |
| | | Surface hydration |
| MCT actuation | Cilia beating | Acoustic vibration |
| MCT direction | Cilia polarization | Janus metal coating, |
| | | 3D printing |

Inspired by the biological MCT epithelium, various aspects of this disclosure are directed to developing engineered surfaces that enable MCT function (i.e., an engineered surface of MCT) with a combination of ciliary structure fabrication, cilia polarization, surface chemical modification, and acoustic actuation (Table 1). Recapitulating ciliary microstructure, surface chemistry, polarization, and beating will enable MCT across the engineered surface. Engineered surfaces of MCT according to various aspects of the disclosure can be widely applied to medical devices to eliminate mucus plugging, mitigate bacterial infection, reduce the care burden, and eventually enhance the life quality of patients.

Various aspects of the disclosure are directed to microstructured polymers that actively clear mucus under gentle conditions. Inspired by biological MCT and low mucus adhesion surfaces, the inventors propose hydrophilic coatings to reduce mucus stickiness, and to employ low-intensity ultrasound to actuate ciliary microstructures. To the best of our knowledge, there are no reports of such engineered surfaces that achieve an active mucus clearance function. In addition, this mechanism can clear mucus under low power input and without using any non-biocompatible chemicals, removing major obstacles for future clinical applications.

The manufacturing approaches support both laboratory and real-world applications. In some aspects of the present disclosure, fabrication methods according to the disclosure utilize a templated-based method to prepare microstructured polymers such as those having ciliary microstructures. With well-controlled size of ciliary microstructures, the mechanism of MCT on engineered surfaces can be realized. Furthermore, engineered MCT surfaces for future real-world applications can be fabricated with 3D printing.

Mucus clearance is assessed with innovative animal models. Rather than proof-of-concept studies that predominantly focus on structure fabrication, MCT is tested in vitro (for example, a slug model) and in vivo. For example, recently developed slug mucus models allow precise control over mucus biophysical properties (e.g., elasticity and viscosity) and shares great similarity to human airway mucus. Pig models can also be used to evaluate engineered MCT surface in vivo. The pig airway best recapitulates human lung physiology, structure, mucus properties, and hallmarks of lung diseases. Moreover, an in vivo test best maintains the physiology of mucus secretion and clearance.

Study design to recapitulate functional elements of biological surface of MCT. A polymeric substrate with polarized cilia microstructures is fabricated using a template-based method or 3D printing method. The ciliary surface is modified to reduce mucus stickiness. MCT is actuated by acoustic waves and tested in vitro using slug mucus and in vivo with pig models. Both genders of pigs in equal numbers can be used in the pig models. SU-8 photoresist templates for microstructures can be fabricated through standard photolithography processes. Engineered MCT surfaces can also be produced via 3D printing processes.

A pig model of cystic fibrosis and its wild type littermates are purchased from Precigen Exemplar and housed in UI Animal Care Unit (see Vertebrate Animal Section). Large terrestrial banana slugs native to the Pacific Northwest region of North America, are purchased from Niles Biological. They are housed and raised in the laboratory at 13° C. Slug mucus vesicles are collected and ruptured using ultrasound at certain vesicle concentrations, pH, and calcium concentrations, to form mucus with a specific value of elasticity and viscosity.

Engineered surfaces without coating, microstructure, acoustic actuation, or polarization are used as control groups when testing mucus stickiness and clearance.

Fabricating Engineered Surfaces with Polarized Ciliary Structures

Engineered MCT surfaces according to various aspects of the disclosure can be fabricated using two complementary methods, template-based methods and 3D printing. Template-based methods allow for fabricating biological cilia sized structures, with precision control of structural geometry and dimensions, and suitable for laboratory test of mucus stickiness and acoustic resonance. On the other hand, 3D printing technology allows for direct fabrication of ciliary structures having complex shapes (e.g., tubular shape for tracheal tube, spherical shape for eye prothesis), and features great manufacturing scalability. Table 2 provides a Comparison between template-based and 3D printing methods.

TABLE 2

| Features | Template method | 3D printing |
|---|---|---|
| Cilia size | Biological cilia-like | Larger than biological cilia |
| Resolution | High (<100 nm) | Medium (′10 um) |
| Overall shape | Plain | Complex geometries |
| Application | In vitro test | In vitro and in vivo tests. |

### Template-Based Fabrication of Engineered Ciliary Surface.

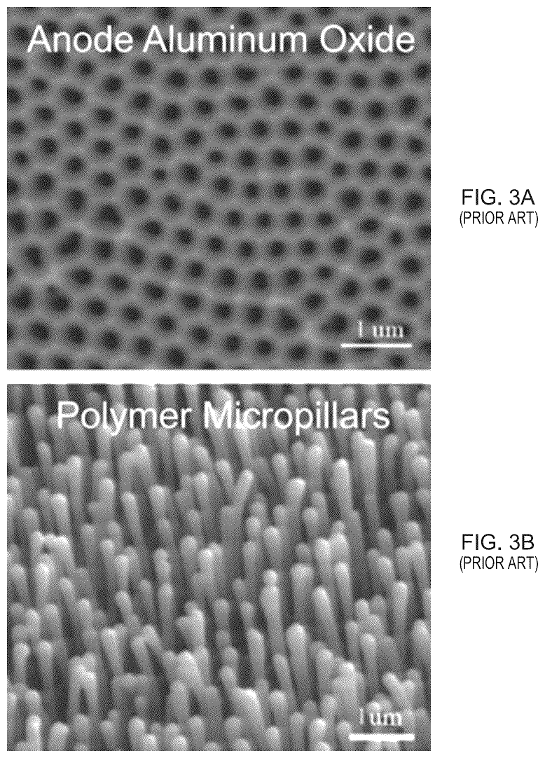

Polymeric ciliary microstructures have been fabricated using template-based methods. For example, self-ordered porous anodized aluminum oxide (AAO) is a preferred embodiment with regularly arranged, parallel nanopores with narrow diameter distribution and uniform depth (FIGS. 3A and 3B). The AAO template has been used to prepare polymer nanopillars of a variety of materials, including poly(dimethylsiloxane) (PDMS), and poly(ethylene glycol) diacrylate (PEGDA). The diameter and length of prepared nanopillars are determined by the AAO template. Other than AAO templates, photolithography process was used to fabricate a SU-8 photoresist template with controlled geometry.

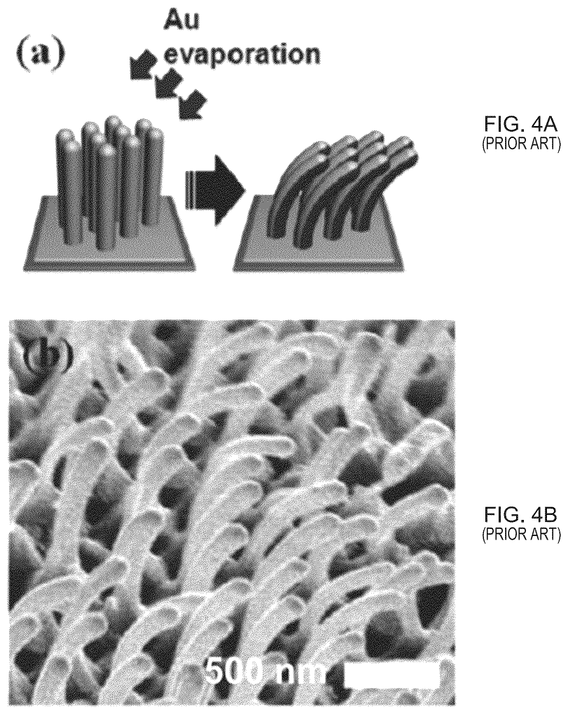

Polymeric microstructures have been polarized. For microstructures fabricated with template-based methods, a conversion from straight nanopillars to uniformly bent Janus nanopillars was achieved by the Janus metal deposition. Here, one lateral side of the polymer nanopillars was coated with gold that was tens of nanometers thick by thermal evaporation (FIGS. 4A and 4B). The Janus nanopillars were bent toward the Au-deposited side, and their curvature was controlled by the thickness of the Au layer. Polarized engineered cilia can be generated to direct the motion of mucus.

Polymers—Two polymers may be preferred for fabrication of ciliary structures. The first is PDMS, due in part to its wide application in microfabrication (e.g., microfluidic devices). The second is PEGDA, due in part to its hydrophilic nature, and extensive application in 3D printing and tissue engineering.

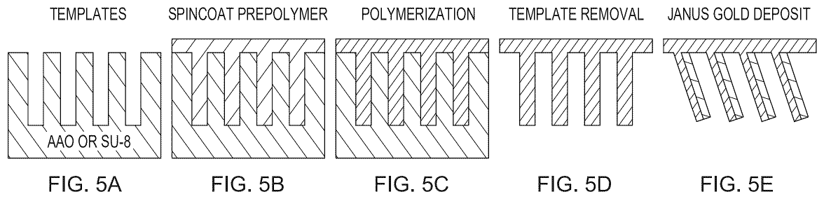

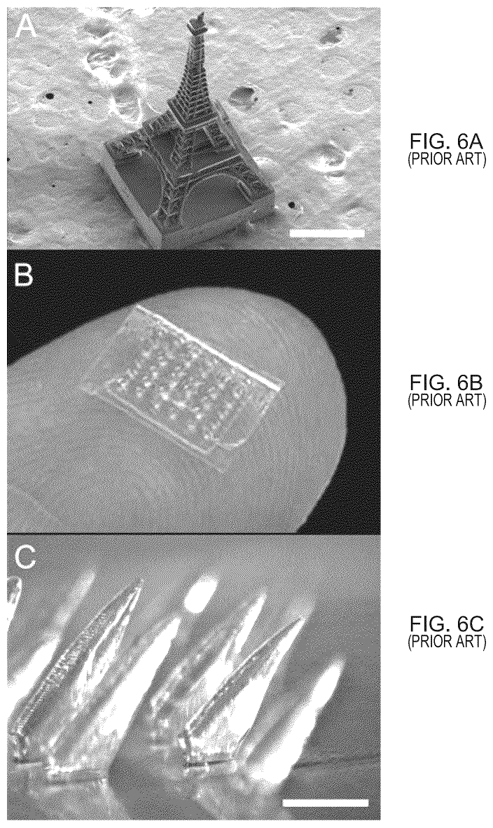

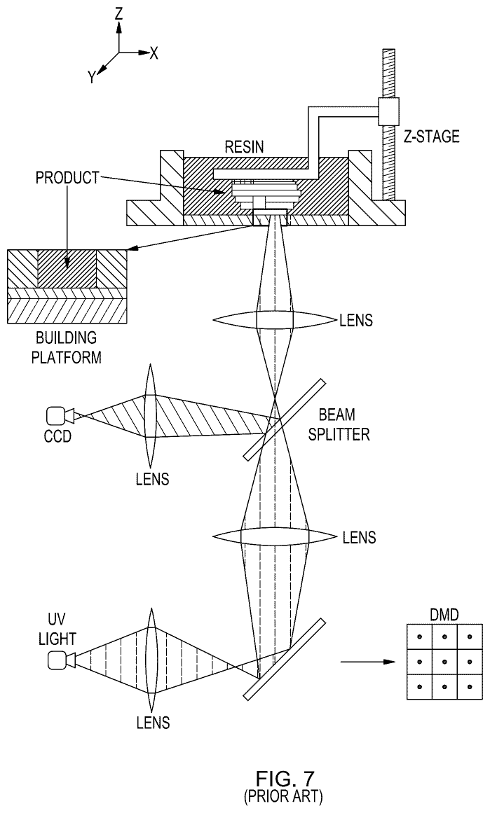

Fabrication of polarized ciliary microstructures—Anodized aluminum oxide (AAO) is used as templates to cast polymer ciliary surfaces (FIG. 5A). Pre-polymer of PDMS or PEGDA are spin-coated on AAO templates (FIG. 5B). After polymerization (heat polymerization for PDMS, and UV polymerization for PEGDA) (FIG. 5C), the template is removed (AAO removal by 0.1M NaOH) (FIG. 5D). A thin layer of polymer containing predefined ciliary structure is harvested. Janus gold-evaporation method is used to polarize the ciliary structure. Here, one lateral side of the polymer nanopillars is coated with gold (thickness ˜10 nm) by tilted E-beam thermal evaporation. The Janus nanopillars are bent toward the Au-deposited side (FIG. 5E).

Various methods, including Scanning Electron Microscopy (SEM) and optical microscopy, can be used to characterize the diameter, length, and polarization of ciliary microstructures.

First, it is expected that the size of ciliary structure is controlled, at least in part, by templates. Size is important for microstructures because it determines the acoustic resonance, which will be used later to actuate mucus motion. The diameter of the AAO pore controls the cilia microstructure diameter, the depth of the hole controls the cilia microstructure length. Of note, commercialized AAO templates have a variety of diameters (50-500 nm) and lengths (300 nm-60 μm). It is expected the ciliary microstructure is polarized with Janus gold deposition. Ciliary polarization determines MCT direction; it is expected that the degree of polarization is controlled by the thickness of gold deposition and the stiffness of the polymer. The Young's modulus of PDMS can be controlled by the ratio of PDMS pre-polymer and curing agent. The Young's modulus of PEGDA is determined by its concentration and molecular weight.

Alternative approaches—First, AAO provides several size choices for fast fabrication of microstructures. To diversify the size and shape of ciliary microstructures on engineered MCT surfaces, photolithography methods with SU-8 templates can be utilized, which allows freedom in designing ciliary microstructures. SU-8 templates can be removed by acetone. Second, the inventors proposed to control ciliary polarization by the thickness of gold coating; alternatively, researchers also incorporated magnetic micro/nanoparticles to polarize the ciliary structure by magnetic field. For example, it is demonstrated that micropillars with 1 μm diameter and 50 μm length could deflect its end up to 12 μm under a gradient magnetic field of 5 mT/mm.

3D Print Medical Devices (e.g. Tracheal Tubes) with Integrated Ciliary Surfaces.

Presented herein are demonstrated 3D prints of microstructures with resolution <10 μm. Others have developed multi-scale projection stereolithography (MPS) processes that can fabricate microstructures with resolution of ˜2 μm on the horizontal XY plane and ˜10 μm on the vertical Z plane, over a large area (˜50 mm*50 mm) (FIG. 6A). Traditional 3D printing processes usually have limitations in building multi-scale objects spanning multiple length scales from micrometer to centimeter, due to the tradeoff between resolution and overall fabrication size. The MPS process overcomes this tradeoff by utilizing a low-cost micro-image scanning strategy in the fabrication. The high resolution, scalable 3D print system has been successfully applied to produce microneedle arrays and bone scaffolds. In the proposed study, the MPS system will be used to 3D print medical devices with microstructure surface for MCT.

Microstructures were created with various or multiple materials. Previously developed MPS systems are compatible with commercial resins from Formlabs, which has various mechanical properties. Furthermore, the MPS systems can directly 3D print multi-material structures, whose physical properties can be tailored via strategically designing the ingredient arrangement. For example, microneedle arrays can be made with two types of materials (FIG. 6B-C), including a rigid polymer as the base and a drug-encapsulated biopolymer as the needles. The process has been modified to achieve fiber-reinforced polymer composites with biomimetic anisotropic fracture toughness through locally changing the orientations of fiber reinforcement. The MPS system is used to fabricate pillars with different stiffness and surface properties.

The MPS system consists of a micro-image projection module, a XY motorized building platform, a resin reservoir, and a motorized Z stage (FIG. 7). The projection module is based on a digital micromirror device (DMD) containing a 1,080×800 array of micromirrors, each of which can be controlled to change at two angles to reflect light onto the building platform. The DMD projected image is resized to a small area of 1 mm×2 mm through objectives and lenses, enabling a resolution of 2 μm on the XY plane. To achieve multi-scale fabrication, The XY motorized platform can be translated along the XY plane. A convolution neural network-based deep learning algorithm was used to control the stitching of the micro-scale images from each scanning location into a large area. To control the growth of feature, a motor driven Z-stage allows Z resolution of m.

Presented herein is 3D-printing a tracheal tube for newborn pigs. Tubular structures (diameter 8 mm and length 30 mm) are fabricated with the MPS system. On the luminal surface, pillar-like structures (length of 200-2000 μm, width of 5-100 μm) are printed. The wide range of size allows the ability test with different acoustic frequencies. A computer-aided design (CAD) model is sliced into a series of binary images with a certain layer thickness (e.g., 10 μm); a liquid resin is purged into the resin reservoir (FIG. 7) by a pumping system; the printing process then starts by sequentially moving the XY motorized building platform to different locations and exposing a micro-image pattern from the sliced CAD model; this process is repeated along Z-direction until all layers are printed. In some instances, two types of photocurable resins can be used, including a commercial clear resin (Formlabs FLGPCL01, USA) and a PEGDA resin. A PEGDA resin is chosen because it is biocompatible and hydrophilic. Sudan I (Sigma-Aldrich, USA) is added in both resins to improve the curing accuracy at micro-scale.

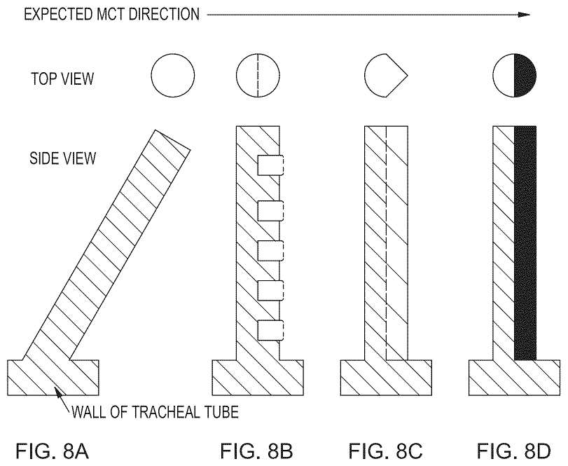

Polarized microstructures are directly formed at the luminal surface of tracheal tube with either direct printing (FIG. 8A), textures along the Z direction (FIG. 8B), cross-section shapes (FIG. 8C), or multi-material prints (FIG. 8D). Microstructures are visualized with microscopes.

3D printing methods are expected to create ciliary microstructures that are larger than biological cilia for MCT. Larger structures are expectedly superior for an engineered surface of MCT. First, larger structures (e.g., ˜10 μm in diameter and ˜200 μm in length) are easier to fabricate than structures with size like biological cilia (0.2-0.3 μm in diameter and 6-7 μm in length), which reduce manufacturing constrains. In addition, because larger structures usually require longer acoustic wavelengths for resonance, and acoustic waves with longer wavelengths have larger penetration depth than acoustic waves with shorter wavelengths, thus more suitable for in vivo applications.

3D printing methods are also envisioned to be able to directly manufacture polarized microstructures. It is expected tilted structures, Z-direction textures, cross-section shapes, and multi-material designs lead to asymmetric vibration and directional flow once actuated by acoustic energy.

Alternative approaches—First, the resolution of 3D printer (2 μm for MPS system) is larger than microfabrication (˜100 nm for AAO template), therefore, the size of 3D printed ciliary microstructure will be larger than microfabricated ones. Considering that resonant frequencies are size dependent; it is expected using acoustic waves with lower frequencies which match the resonant frequency (described in Aim 3). Nonetheless, current commercial 3D printers can be adopted to manufacture medical devices with MCT functions. For example, Formlabs produces a Form 3+ printer featuring resolutions of 25 μm.

### Mucus Stickiness on Engineered Surface of MCT

Biological surfaces of MCT have macromolecules-tethered cilia membrane and periciliary liquid layer to prevent mucus adhesion. However, engineered surfaces of in-use medical devices lack these features, thus how these factors effect mucus-surface interactions (i.e., mucus stickiness) remains largely unknown. Various surface chemistries of ciliary microstructures can be fabricated and evaluated for the tendency of mucus to stick on or adhere to engineered surfaces of MCT. It is expected that a hydrophilic ciliary surface reduces mucus stickiness. Biological ciliary surfaces contain many tethered macromolecules, such as polysaccharides. Consequently, mucus cannot penetrate into the periciliary space, which forms a distinct mucus-less periciliary liquid layer. Periciliary liquid prevents the epithelium from adhering to the overlying mucus. Coatings of hydrophilic molecules reduce mucus adhesion.

Uncoated pillars of PDMS are hydrophobic. An extensive number of reports describe the coating on PDMS to manipulate surface properties, such as wettability and cell adhesion properties. Since mucus tends to be stickier on surfaces with hydrophobic groups, modifying the surface wettability of PDMS is envisioned as a solution. The inventors also modified surface properties of 3D printed structures. For example, prior studies have modified the surface of 3D printed scaffolds for healing critical bone defects. Osteo-conductive scaffolds printed by MPS technology were coated with different growth factors to achieve spatially differential bone regeneration. The results have shown that surface coating of 3D printed structures can significantly modify the physicochemical interaction between the 3D printed structures and surrounding environment. Similar surface modification procedures were commonly used for 3D printed biomedical devices. Various engineered surfaces disclosed herein exhibit a reduced tendency to stick to mucus.

Various strategies can be used to reduce mucus stickiness on surfaces with engineered ciliary structures. First, engineered surfaces can be coated with a polyethylene glycol (PEG) or a glycan. PEG can be coated by direct dipping ciliary surfaces into a PEG solution. It has been reported that PEG reduced interaction between mucus and coated materials, and PEG has been used for mucus-penetrating nanoparticles. In addition, PEG is hydrophilic, which allows a layer of liquid between ciliary structure, i.e., periciliary liquid. Glycans can also be coated onto PDMS surfaces, to mimic the cell surface glycans. Alternatively, PEGDA ciliary structures can also be fabricated as described elsewhere herein.

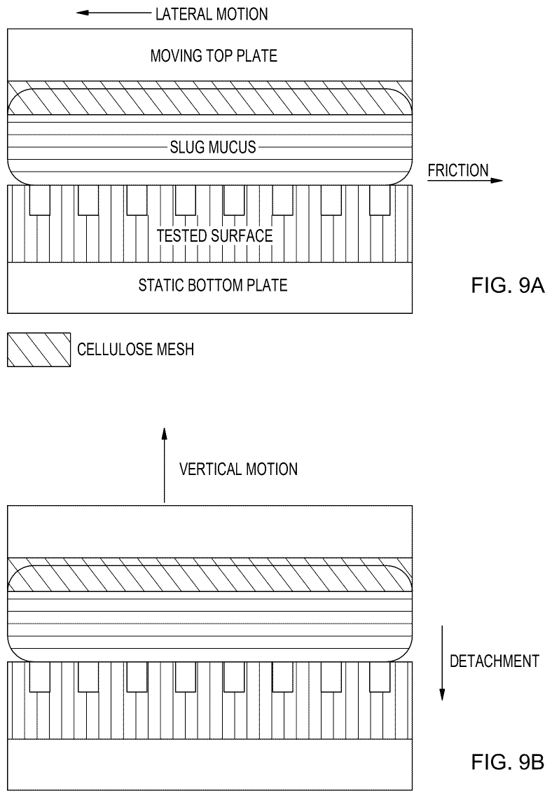

Mucus stickiness/adherence to engineered microstructures can be evaluated by measuring friction (i.e., the force against lateral motion of mucus) and detachment (i.e., the force against detaching mucus from surface) with a Tribo-Rheometer, which is commonly used to measure surface interactions. In this experiment, slug mucus is attached on the top plate of a Tribo-Rheometer, where the plate is coated with a cellulose mesh to anchor mucus. The bottom plate will be placed with the tested surface with microstructures. To test the friction between mucus and ciliary surface, lateral motion of the top plate is applied (FIG. 9A). To test the detachment force between mucus and ciliary surface, vertical motion of the top plate is applied (FIG. 9B). The force response is recorded with respect to the displacement and velocity of the top plate.

Ciliary microstructures or microstructured polymers coated with hydrophilic coatings such as, for example a PEG or a glycan, are expected to exhibit reduced friction and attachment of mucus. Mucus tends to be stickier on surfaces with hydrophobic groups, and theoretical studies confirmed this affinity is due to surface energy. If a reduced stickiness with hydrophilic coatings is observed, it will confirm that the macromolecular tethered cilia membrane is a key element to repel mucus from the ciliated epithelium. As confirmation, plasma would be used to treat PDMS surfaces, which transiently (last ˜30 minutes) changes the surface hydrophilicity without macromolecules, and mucus stickiness will be evaluated.

It is also expected that, once the surface is coated, mucus stickiness is not determined by geometries of microstructures. If so, it would suggest that ciliary structure only acts in actuating and directing MCT, but mucus stickiness is mainly determined by the surface chemistry. This outcome would also allow more freedom to design the geometry of microstructure without being concerned with effects on stickiness. Alternatively, it could be observed that microstructures reduce the stickiness of mucus, because it reduces the area of contact between mucus and the surface. If this is true, further tests with structures of different sizes will be run, which can be fabricated with different templates. The surface structures are less likely to impact mucus stickiness, because many biological mucosal surfaces are not sticky to mucus and not ciliated (e.g., intestine and gallbladder).

Through non-specific absorption, a PEG or glycan coating can be applied to the PDMS. Alternatively, if there isn't sufficient coating on a target surface, a PEG-thiol can be used instead, which bonds to gold via gold-sulphur interface, while not affecting the PEG-PDMS electrostatic interactions. To reduce mucus stickiness, the surface can be coated with a PEG or glycan. Alternatively, features can be directly printed with mucus non-sticky materials onto the PDMS.

### Developing Platforms to Test Acoustic Actuated MCT on Engineered Surfaces.

Rationale. In biological MCT, mucus is actuated by beating cilia and directed by cilia polarization. On the other hand, there is a lack of platform to investigate or apply the mechanism of MCT on engineered surfaces. Herein, platforms have been developed to test acoustic actuated mucus motion on engineered surfaces of MCT. An in vitro system is developed, where acoustic wave is used to vibrate polarized, surface modified microstructures to promote movement of slug mucus. MCT is tested in vivo by placing a tube with luminal surface of MCT into the trachea of a newborn pig with cystic fibrosis (CF). Pigs are utilized because they best recapitulate human airway physiology, structure, and mucus properties.

Banana slug models are used herein to model human mucus. The mucus producing tissue of banana slugs resembles that of mucus cells in human submucosal glands. Lectin staining and scanning electron microscopy show that pig and slug mucus are similar. More importantly, intact mucus vesicles can be readily collected and ruptured under laboratory conditions to form mucus. Notably, the biophysical properties of slug mucus (e.g., viscosity, elasticity, wettability) can be adjusted by the chemical environment during the mucus formation. Slug mucus will be used to test the engineered MCT surface in vitro. With CF, their airways produce copious mucus under methacholine stimulation.

CF pigs are developed as a model for human airway disease. A pig model with disrupted genes encoding cystic fibrosis transmembrane conductance regulator (CFTR) anion channel was developed in Iowa. The pig model recapitulates human airway anatomy and spontaneously develops hallmark features of CF lung disease, including mucus accumulation and infection. Furthermore, under methacholine stimulation, copious amounts of mucus accumulates on the CF trachea. In addition, newborn pigs eliminate the effects of secondary manifests such as infection and inflammation, which might cause variations of mucus production. The newborn CF pigs, and their non-CF littermates, provide a model to test devices with engineered surfaces of MCT in vivo.

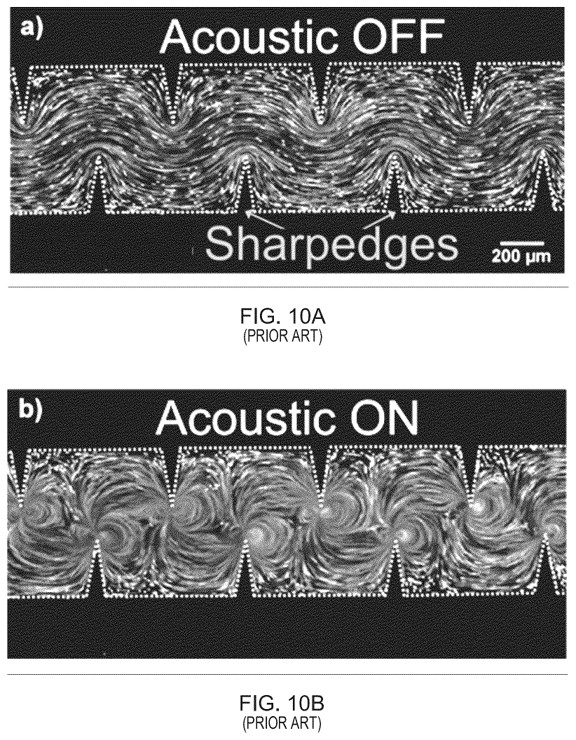

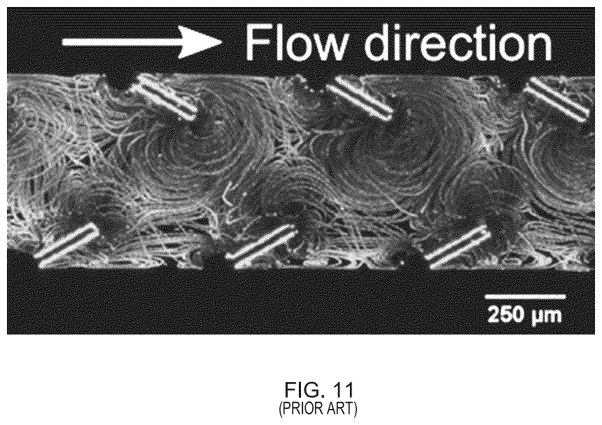

Engineered microstructures developed herein can be subjected to acoustic waves to direct flow. Under acoustic wave actuation, the microstructures perturb the surrounding fluids (FIGS. 10A and 10B), achieving rapid, homogeneous mixing. Furthermore, under acoustic actuation, tilted sharp-edges (FIG. 11) generate directional flow, forming a micro-pump in a microfluidic channel. This sharp-edge-based acoustic pump can generate stable flow rates as high as 8 μL min⁻¹, which are tunable across a wide range (nL/min to μL/min). The proposed ciliary structures share similar polymer material, fabrication approach, and actuation mechanism of microfluidic sharp-edges, and will be used to actuate MCT on engineered surfaces.

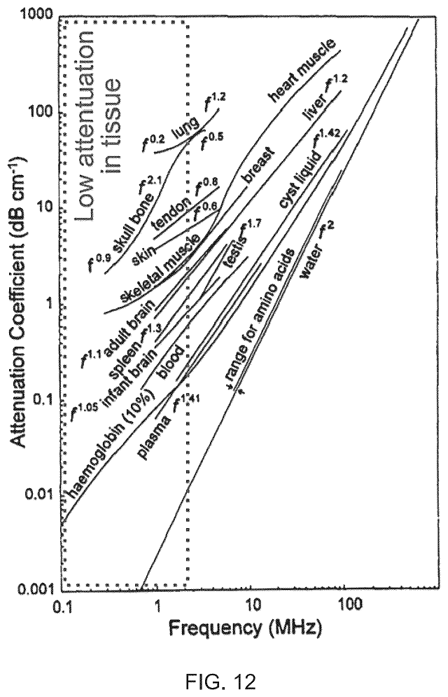

Acoustic waves can penetrate hydrogel, human tissue, and tissue-device interfaces. When acoustic waves are transmitted in a complex medium composed of multiple materials, the wave intensity can be reduced both within the material and at the interfaces, due to absorption, reflection and scattering of acoustic waves. These factors do not prevent acoustic actuation of microstructures according to the disclosure. First, acoustic waves transmit efficiently through homogeneous, elastic, soft materials. Attenuation coefficient quantifies how much energy is weakened by the material it is passing through. It is showed that low MHz ultrasound has acoustic attenuation coefficients <1 dB/cm in collagen hydrogels. In addition, kHz or low MHz frequency ultrasound maintained most of its energy when passing soft human tissues, as demonstrated by low attenuation coefficient (boxed in red, FIG. 12). Second, the transmission coefficient is high at material interfaces. For example, if agar gel and PDMS microstructure are used, the interfacial transmission coefficient (TAgar-PDMS) is calculated using Eqn. 1. Since acoustic impedances of PDMS (ZPDMS) and agar gel (Zagar) are similar, the acoustic wave intensity is transmitted 96% at the interface. The low attenuation sets foundational basis for in vivo acoustic application including ultrasound imaging, kidney lithotripsy, particle manipulation, and this proposed work.

In vivo acoustic manipulation is demonstrated experimentally and clinically. Acoustic wave was recently used to manipulate kidney stones or particles in vivo. For example, Ghanem et al. precisely positioned glass particles in pig bladder in vivo with an array of ultrasound transducers at frequency of 1.5 MHz (FIG. 13). Harper et al. showed the first-in-human clinical trial of ultrasonic (350 kHz frequency) propulsion of kidney stones with no adverse effects on patients. Experimental successes encouraged us to pursue mucus actuation on MCT surfaces in vivo.

$${T}_{Agar - PDMS} = 1 - {[ \frac{{Z}_{Agar} - {Z}_{PDMS}}{{Z}_{Agar} + {Z}_{PDMS}} ]}^{2} = 96 \% , \\ where {Z}_{PDMS} = 1.05 \times {10}^{6} \frac{kg}{{sm}^{2}} , \\ {Z}_{Agar} = 1.57 \times {10}^{6} \frac{kg}{{sm}^{2}}$$

### In Vitro Assessment of Mucus Motion on Engineered Surface of MCT

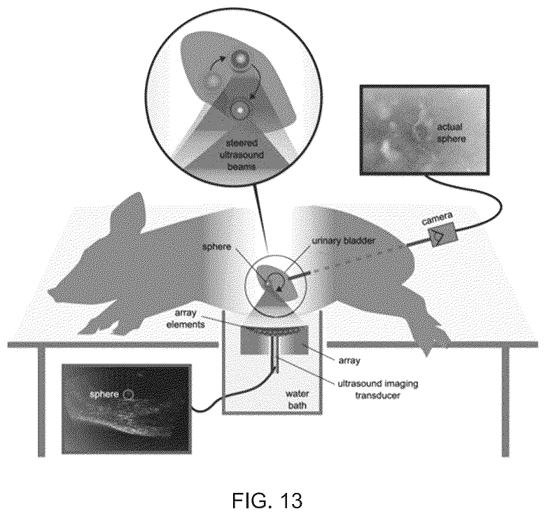

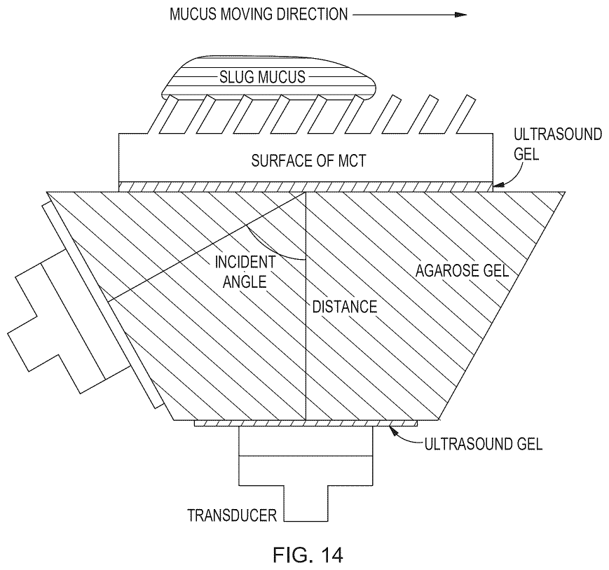

The setup comprises a piece of ciliary MCT surface, made by either PDMS microfabrication or 3D printing placed on top of agar gel (FIG. 14). The ciliary microstructures are polarized and modified to reduce mucus stickiness. Agar gel is used to mimic human tissue, because agar gel has similar stiffness, density, and therefore speed of sound as tissue. Slug mucus is applied onto the surface of MCT. On the other side of agar gel, a piece of transducer (kHz-low MHz, Steiner & Martins, USA) is attached to generate acoustic wave. The transducer is actuated by function generator (E4422B, Agilent, USA) and amplifier (100A250A, Amplifier Research, USA), with varied output power levels (100 mV-10 V). The distance and incident angle of transducer can be adjusted. To prevent loss of acoustic energy, ultrasound gel (McKesson, USA) is applied at the interface of MCT surface-gel and gel-transducer.

Quantitative assessment of MCT velocity: Fluorescent microparticles (5 μm) are applied in mucus gel; thus, the speed and direction of mucus motion can be quantitatively assessed by particle tracking. To understand MCT velocity, the speed of particles at varied acoustic wave power levels is plotted; the direction of particle motion versus ciliary polarization is plotted.

Resonant frequency of microstructures: Transducers with different resonant frequencies (Steiner & Martins, USA) are tested. Each transducer will sweep the frequency of acoustic signal, and using high frame-rate camera to continuously monitor the cilia vibration.

Acoustic penetration through agar gel: The thickness of agar gel is adjusted from 0 (mimicking no tissue barrier) to 50 mm (mimicking the maximum neck tissue thickness) to test acoustic attenuation in tissue. The incident angle is adjusted from 0 to 60 degree to test direction of acoustic wave impacting mucus motion. Acoustic wave is applied with identical frequency and amplitude. The speed of particles motion vs gel thickness is plotted.

Expected results and interpretation: It is expected acoustic waves will vibrate engineered (ciliary) microstructures and perturb surrounding fluids. A previous study showed that pillar-shaped structures can be actuated with acoustic wave and develop acoustic streaming around the microstructure. The acoustic streaming was used to mix, pump, and induce sputum liquefaction reactions. The belief is that that the acoustic streaming is very likely to happen around ciliary structures here. Furthermore, it is expected that for a microstructure with a specific dimension, its vibration can be maximized at resonant frequencies. All vibrating objects resonate at particular resonant frequencies, depending on the material, geometry, and mode of oscillation. Based on the principles of acoustic resonance, the cilia tips vibrate most when cilia length is ¼ of the acoustic wavelength. Particularly, it is envisioned that larger structures have lower resonant frequencies. Considering larger structures can be manufactured with 3D printing, and longer acoustic wavelengths penetrate more in tissues, larger structures may be better than natural cilia-sized microstructures for MCT.

Second, it is expected that the direction of mucus motion is controlled by cilia polarization, not acoustic wave incident direction. In biological MCT, the direction of mucus movement is guided by the directional growth and vibration of cilia. In disease with pathological cilia development (e.g., primary ciliary dyskinesia), MCT is impaired. It has already been demonstrated that actuating polarized microstructures in a microfluidic produces a directional flow in microfluidic channels. In addition, a further expectation is that the direction of acoustic waves does not determine mucus motion direction, which can be evaluated by changing the relative positioning of an acoustic transducer and tracking particles (FIG. 14). In addition, it is envisioned that the speed of mucus motion is controlled by acoustic power. Previously, it was observed that changing the acoustic input power could tune the pumping flow rate when a vibrated microstructure in used. Here, it is expected that the speed of particle motion is positively related to the acoustic power input. If so, it would suggest that one could rationally adjust the clearance of mucus by changing the acoustic power.

It is also expected that acoustic waves can penetrate agar gel with ˜5 cm thickness to actuate MCT. 5 cm is the estimated largest distance from the luminal surface of tracheostomy tube to the surface of body, suggesting that this strategy has potential for clinical application. In fact, both theoretical calculation and clinical in vivo application of low-MHz ultrasound suggest the success.

In Vivo Test of Mucus Clearance on a Tracheal Tube with Engineered Surface of MCT.

Animal preparation: Pigs are anesthetized with ketamine and xylazine, and sedation is maintained with propofol (see Vertebrate Animal Section). Pigs breathe spontaneously, allowing us to perform the experiment for ˜4 hours. Animals are placed in an enclosed humidified chamber (100% relative humidity, at 25-30° C.) for experiments.

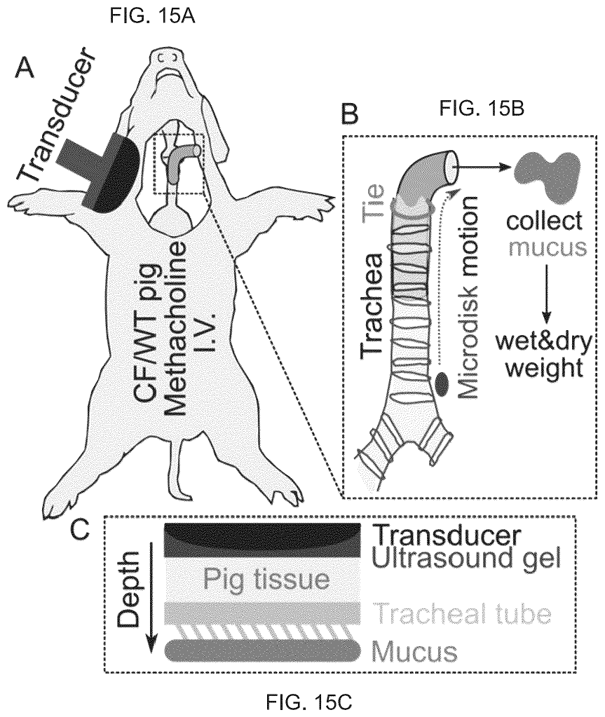

Tracheal tube placement (FIG. 15A). A tracheostomy tube with the luminal engineered surface of MCT is 3D printed as described above. The neck of pig is dissected to expose the trachea. A small incision is made in the ventral tracheal wall to place the tracheostomy tube. The fabricated tube is inserted into the pig trachea. The trachea and device are tied together to prevent leaking. After stitching the skin of pig neck, mucus secretion is stimulated with methacholine (2.5 mg/kg, i.v.).

Acoustic system: kHz-low MHz acoustic waves can be generated by two strategies. First, acoustic waves can be generated using a clinical applied ultrasound generator. For example, earlier work used a Vantage™ Research Ultrasound System (Verasonics, USA) and HDI C5-2 transducer array (Philips, USA) to move kidney stones in humans and pigs. Alternatively, a customized acoustic system could be used. Of note, an earlier study used similar customized setups to manipulate particles in pig urinary bladders. To apply acoustic waves, the pig neck is shaved clean before the acoustic operation. A transducer probe or disk is placed on the pig skin. Ultrasound gel (McKesson, USA) is applied to eliminate any air between probe and tissue, allowing maximum acoustic wave transmission (FIG. 15C). This procedure reduces the distance between transducer and MCT surface to a layer of tissue of 10 mm thickness.

Quantitative assessment of mucus clearance: To assess amount of mucus being cleared from tracheal tube, mucus is collected at the end of the tube with cotton tips (FIG. 15B). The wet weight (i.e., mucin, protein and water) and dry weight (i.e., mucin and protein) of mucus are measured. To understand the velocity of particle motion in the tube, tantalum microdisks are applied into the tube. The particle motion is tracked by an x-ray computed tomography-based assay. Mucus clearance is compared with or without ciliary surface, acoustic actuation, and under varied environmental humidity on both CF and non-CF pigs.

Injury assessment: Pig tissue around the acoustic impacted area is sectioned, fixed, and stained with hematoxylin and eosin for injury assessment.

Expected results and interpretation: First, it is expected that, under acoustic actuation, the tracheal tube with the luminal surface of MCT increases the weight of mucus being cleared. If so, it would indicate that acoustic actuated MCT could transport both water and mucin. It is also expected that the moving speed of tantalum microdisks, characterized by x-ray scanning, is regulated by the level of acoustic power. This is expected because it has been previously demonstrated that the flow rate of sharp-edge enabled micropump is proportional to acoustic wave intensity.

Second, it is expected that the tracheal tube with the luminal surface of MCT increases mucus clearance for both non-CF and CF pigs. Although CF is characterized by defective MCT (FIGS. 2A and 2B), this defect is because the mucus does not break normally from submucosal glands, rather than transport of mucus on airway surfaces. In CF, once a mucus strand is released, the velocity of mucus motion on airways is not different from non-CF. This result indicates motion of mucus on surface of MCT is independent of mucus biophysical properties. If, in fact, both CF and non-CF mucus pass readily through the presented engineered MCT airway surface, then the presented MCT device should be suitable for patients with diseased mucus as well as normal mucus.

Third, it is expected that the MCT tracheal tube will maintain its function with environmental hydration. Mucus is not sticky to the surface of MCT because the hydrophilic surface traps a thin layer of liquid and repels mucus. If the MCT tube directly contacts the humidified air, the inventors predict that surface hydration will be maintained, and mucus will move readily with acoustic ciliary vibration. MCT function is expected to be lost because of dehydration of engineered cilia in a de-humidified environment.

Fourth, it is expected that tissue injury should not be a complication of acoustic manipulation. It is very unlikely that acoustic actuation will harm overlying tissue since previous research on acoustic in vivo manipulation, under similar frequencies and energy levels, showed great tissue intactness.

Alternative approaches: First, the tracheal tube might lose MCT function in a dehydrated environment, because when the mucus-free liquid layer dries, mucus will begin accumulating on the device. This is expected to be resolved by rehydrating the surface. If the surface coating still functions after rehydration, then the MCT function can be regenerated. Second, the interface between tissue and the MCT device might impact the MCT from trachea to device. Here, the engineered MCT tube can be inserted into a pig trachea and tied together to eliminate gaps between the two. In future clinical applications, approaches can be taken (e.g., designing the edge of device, or cleaning approach on the end of device) to facilitate mucus transport across trachea-device interfaces. Third, current acoustic systems are expensive, which could be a barrier for future practical applications. To this end, if the resonant frequency of MCT microstructures is properly designed, it could be actuated by an inexpensive speaker and controlled by a cellphone, which is demonstrated in other acoustic manipulation settings. If achieved, the potential for bedside application of MCT devices is enormously expanded.

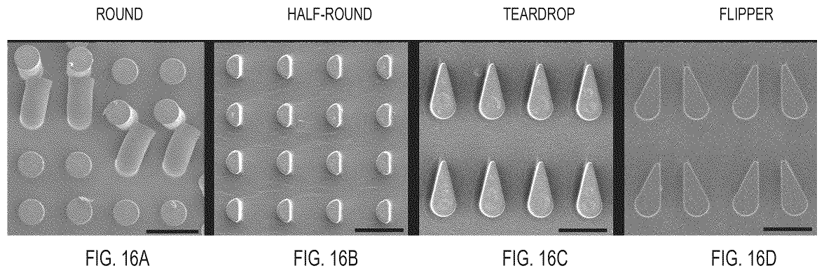

First, designed and fabricated microstructures are used to actuate fluid motion. Inspired by the biological ciliary surface, polarized microstructures that would generate directional flow under acoustic waves are designed. To ensure robust and unbiased results, designed arrays of ciliary structures with 4 different cross-sectional shapes (i.e., round, half-round, teardrop, and flipper shape) in 2 sizes (i.e., big, and small), with a total of 8 designs are used. A photolithography process, with photoresist of AZ125 of 70 μm thickness, was applied to prepare a mold of ciliary microstructures. A soft-lithography and mold-replica process was used to prepare polydimethylsiloxane (PDMS) arrays of these structures. Principally, liquid PDMS prepolymer and curing agent were mixed with a ratio of 10:1 (w/w). The mixture was poured onto the silane treated AZ125 mold and de-gassed with vacuum pump. After PDMS curing in room temperature overnight, the PDMS layer was carefully peeled off from the mold to generate PDMS pillars. Then, the PDMS pillars were examined by scanning electron microscope (SEM) and used for acoustic fluid transport experiment. The SEM images of PDMS microstructure were demonstrated in FIGS. 16A through 16D.

Vibrating Ciliary Arrays and Generating Directional Flow with Acoustic Wave.

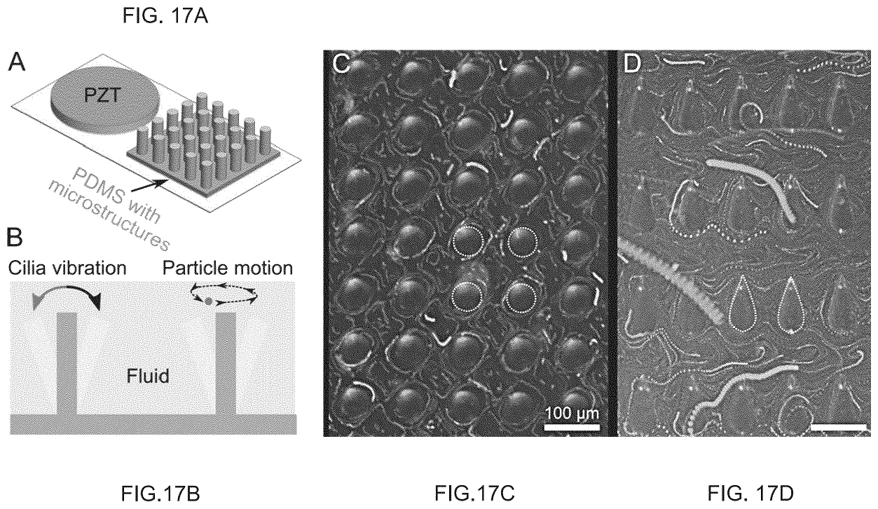

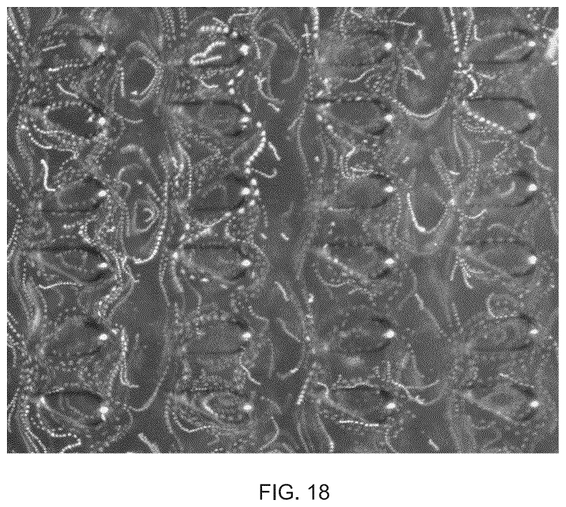

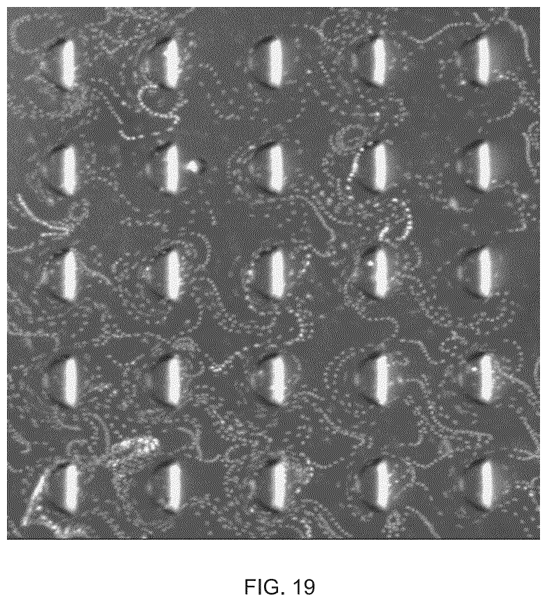

To actuate the vibration of PDMS microstructures, acoustic waves were generated by plate piezo transducers (FIGS. 17A-B). The transducer and the PDMS microstructure surface were bonded on the same glass slide via double-sided tapes. The PDMS ciliary surface was treated with plasma for hydrophilicity. Water was dropped onto the microstructure surface for actuation. Fluorescent microparticles (1 μm) were applied into water to characterize vibration of cilia-like structures and the motion of particles upon acoustic actuation. The PDMS pillars were acoustically actuated at ˜5 kHz frequency. Upon acoustic actuation, fluid around the pillar structure is perturbed, generating acoustic microstreaming. It is observed that ciliary structures with round-shape cross section do not produce a net flow. Instead, a localized vortex is formed between 4 adjacent pillars (FIG. 17C). This is because that round-shape is symmetric in all directions, and it is not possible to achieve an asymmetric acoustic streaming. In contrast, structures with asymmetric cross section shape (i.e., teardrop) generated a net directional flow upon acoustic actuation. The net flow direction is from left to right in FIG. 17D. Additional fluorescent net flow images can be seen in FIG. 18 and FIG. 19. FIG. 18 has pillar structures in the shape of sideways teardrops, and FIG. 19 has pillar structures in the shape of the letter “D”. FIG. 20 presents alternative asymmetrical shapes that can be used for pillar structures. To ensure robust and unbiased results, the inventors rotated 90 degrees of the PDMS pillar surface to confirm that the flow arises from pillar array, rather than relative location between the piezo transduction and PDMS. It was found that the direction of net flow always follows the pattern configuration. That discovery set the basis of this project, that is, by rational design surface microstructure, directional flow is feasible under acoustic actuation.

3D Printed Structures and their Application in Actuation

The inventors have fabricated cilia-like, tilted pillars using 3D printing method. In a representative design, the diameter of each pillar is 200 μm, the length of each pillar is 2 mm, the center-to-center distance between pillars is 1 mm. The pillars form an array that covers an area of 10 mm by 10 mm. The pillar array was 3D printed using a Form 2 printer (Formlab, USA) with flexible photopolymer resins (FLFLG02). It was showed that the high aspect ratio (10:1) pillars were successfully printed on a flat base (FIG. 21). Structures can be 3D printed with different cilia length, diameter, gap, tilting angle, or their combinations.

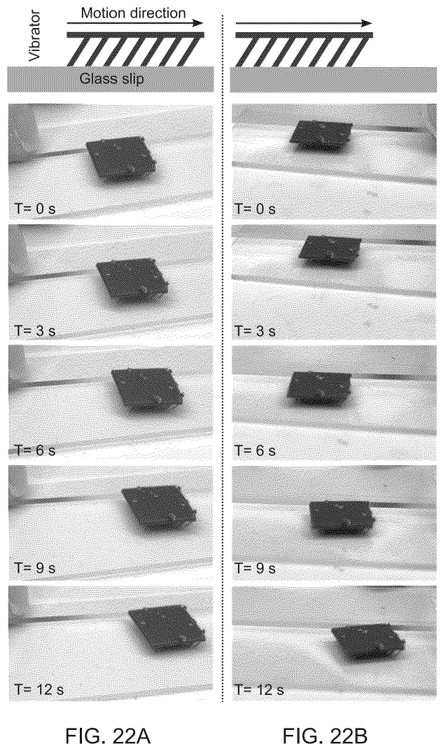

The inventors have demonstrated that, upon vibration, the tilted, cilia-like structures generate force, and the direction of force is determined by the design of structures. In an experiment, the microstructures and their base were flipped up-side-down, so that the cilia-like structures become the “feet” to support the base (FIGS. 22A and 22B). The platform was then placed onto a glass slide that bonded with a vibration motor (Model 1020, Marhynchus, China). The vibrator was connected to a DC voltage generator (SPD3303X-E, Siglent, USA). Upon actuation of vibrator with 2-3 V, the platform with cilia-like structures moved towards the direction of cilia polarization (FIG. 22A). To ensure robust and unbiased results and confirm that the direction of motion is determined by the cilia polarization, rather than the relative position between vibrator and platform, the platform was turned 180-degrees. It is found that the direction of platform motion also turned by 180-degrees (FIG. 22B). In addition, the inventors tested the motion of a platform with vertical cilia-like structures and found that it cannot generate directional motion.

The actuation of tilted, cilia-like structures to direct flow is also envisioned. In an experiment, the platform with tilted, cilia-like structures was bonded in the bottom of a petri dish and submerged with water with a thickness of ˜2 mm. Fluorescent microparticles were added in the water to indicate the fluid motion. Upon actuation of a vibrator with ˜3 V, fluid motion was observed.

Microfabricated Structure and their Application in Actuation

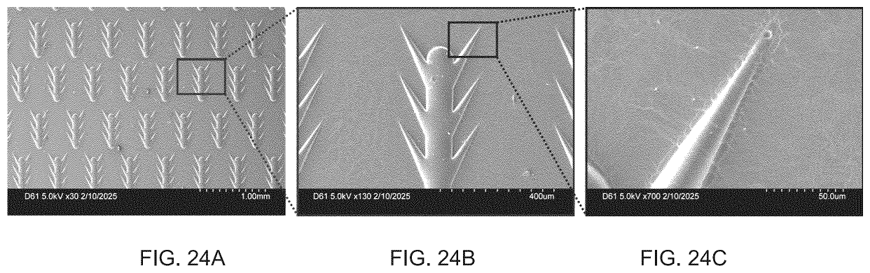

The inventors have designed and fabricated microstructures to actuate fluid motion. An object of the invention is the design of polarized structures that generate directional flow under acoustic waves, such as those illustrated in FIGS. 23A-23E. The mask of these designs were fabricated at a microfabrication facility at University of Minnesota. A photolithography process, with photoresist of AZ125 of 70 μm thickness, was applied to prepare mold of the ciliary structures. A soft-lithography and mold-replica process was used to prepare polydimethylsiloxane (PDMS) arrays of these structures. The representative SEM images of PDMS microstructure were demonstrated as well (FIGS. 24A-24C), where the sharp edges were expected to perturb flow upon acoustic actuation.

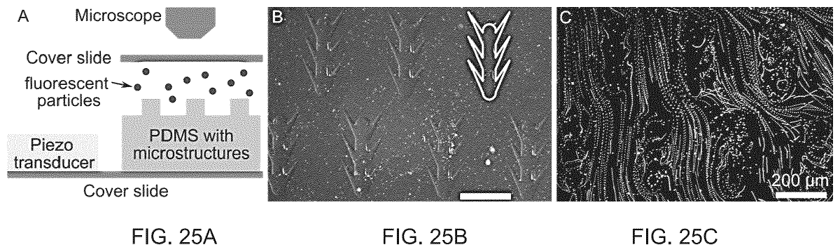

The inventors have vibrated the ciliary arrays and generate directional flow with acoustic wave. To actuate the vibration of PDMS microstructures, acoustic waves generated by plate piezo transducers were used (FIG. 25A). The transducer and the PDMS microstructure surface were bonded on the same glass slide via double-sided tapes. The PDMS ciliary surface was treated with plasma for hydrophilicity. Water was dropped onto the microstructure surface for actuation. To control the depth of water on top of the microstructure, a cover slide was used to coat the water, with the resulted depth of water being ˜150 μm. Fluorescent microparticles (1 μm) were applied into water to characterize vibration of cilia-like structures and the motion of particles upon acoustic actuation. The PDMS pillars were acoustically actuated at ˜5 kHz frequency. Upon acoustic actuation, fluid around the pillar structure is perturbed, generating acoustic microstreaming (FIG. 25B). The trajectories of particle motion showed that asymmetric structures generated a net directional flow upon acoustic actuation (FIG. 25C). To ensure robust and unbiased results, the inventors adjusted the relative position between the micro-structured surface and the piezo transducer and obtained the similar flow direction governed by the structure direction.

3D-printed or microfabricated structures according to the disclosure show that, by rational design surface microstructure, directional flow of a fluid, such as mucus, on a polymeric structure can be accomplished under acoustic or vibrational actuation. The 3D-printed, tilted, cilia-like structures can generate force with a direction controlled by the cilia polarization, which set the basis of using the tilted structure to actuate mucus and fluids. In addition, this experiment confirmed that the direction of force is independent of position of actuator. This is a great advantage of the method because in the real application, the actuator will be used outside of the body, and its relative position with the micro-structured surface is difficult to control.

What is claimed is:

1. A medical device comprising:
a substrate; and
a plurality of microstructures provided on a surface of the substrate, the plurality of microstructures providing the medical device with a microstructured surface.

2. The medical device of claim 1, wherein the substrate is made of a first polymeric material.

3. The medical device of claim 1, wherein the plurality of microstructures is made of a second polymeric material.

4. The medical device of claim 1, wherein the substrate is made of a polymeric sheet.

5. The medical device of claim 1, wherein the substrate is in the shape of a tube, a flat surface, a curved surface, or a sphere.

6. The medical device of claim 1, wherein each of the plurality of microstructures are asymmetrical, as indicated by dissecting one of the microstructures with a vertical plane extending through a center point of the microstructure.

7. The medical device of claim 1, wherein a width or diameter of each microstructure ranges from about 25 nm to about 100 μm.

8. The medical device of claim 1, wherein a width or diameter of each microstructure ranges from about 100 μm to about 500 μm.

9. The medical device of claim 1, wherein the length of each microstructure ranges from about 200 μm to about 2000 μm.

10. The medical device of claim 1, wherein the length of each microstructure ranges from about 40 μm to about 60 μm.

11. The medical device of claim 1, wherein the plurality of microstructures further comprise a hydrophilic coating thereon.

12. The medical device of claim 11, wherein the coating is made of a polyethyleneimine, a polyvinyl alcohol, or a polyethylene glycol.

13. The medical device of claim 1, wherein the plurality of microstructures are polarized and/or asymmetric.

14. The medical device of claim 1, wherein the medical device is associated with a surface of a second medical device.

15. The medical device of claim 1, wherein the second medical device is a tube.

16. A method of removing a fluid from a surface of a medical device according to claim 1, the method comprising:
subjecting the medical device to an energy input to vibrate the plurality of microstructures, wherein vibration of the microstructures induces a flow of a fluid located on the microstructured surface.

17. The method of claim 16, wherein the fluid is mucus.

18. The method of claim 16, wherein the energy input is acoustic.

19. The method of claim 16, wherein the medical device configured for insertion in a body part of a mammal.

20. The method of claim 19, wherein the body part is a trachea.
